(19) United States
(12) Patent Application Publication
(10) Pub. No.: US 2023/0104117 A1
(43) Pub. Date: Apr. 6, 2023

(54) INFORMATION PROCESSING APPARATUS, METHOD, AND NON-TRANSITORY COMPUTER READABLE MEDIUM

(71) Applicant: NEC Corporation, Minato-ku, Tokyo (JP)

(72) Inventors: Daniel Georg ANDRADE SILVA, Tokyo (JP); Yuzuru OKAJIMA, Tokyo (JP); Kunihiko SADAMASA, Tokyo (JP)

(73) Assignee: NEC Corporation, Minato-ku, Tokyo (JP)

(21) Appl. No.: 17/795,948

(22) PCT Filed: Feb. 13, 2020
(86) PCT No.: PCT/JP2020/006653

§ 371 Date: Jul. 28, 2022

(51) Int. Cl. G16H 40/20; G06N 7/00

(52) U.S. Cl. CPC G16H 40/20; G06N 7/005

(57) ABSTRACT

An information processing apparatus for determining a threshold on classification scores includes: a score ranking component that sorts all classification scores from samples of an evaluation data set that was not used for training the classifier and removes scores for which the class label is false; and an iteration component that iterates the threshold from the highest score returned from the score ranking component down until the number of samples with score not lower than the current threshold is larger than a user specified recall value times the number of true labels in the evaluation data set.

### TECHNICAL FIELD

The present invention relates to an information processing apparatus, a method, and a non-transitory computer readable medium for determining a threshold for class label scores such that the expected recall of the classier is above a user-defined value.

### BACKGROUND ART

In many situations, classification accuracy can be improved by collecting more covariates. However, acquiring some of the covariates might incur costs. As an example, the diagnosis of whether a patient has either diabetes or not is considered. Collecting information (covariates) such as age and gender are almost at no cost, whereas taking blood measures clearly involves costs (e.g. working hour cost of medical doctor).

On the other hand, there is also a cost of wrongly classifying the patient. There are two types of misclassification. First, the patient may be classified as having no diabetes, although the patient is suffering from diabetes. The resulting cost is called the false negative misclassification cost which is denoted as c_{1,0}. Second, the patient may be classified as having diabetes, although the patient is not suffering from diabetes. The resulting cost is called the false positive misclassification cost which is denoted as c_{0,1}.

Defining both misclassification costs c_{1,0} and c_{0,1} is crucial for rational decision making which is based on a Bayes procedure. A Bayes procedure for binary classification is defined as follows

$$δ \left(\text{x}\right) = {\text{argmin}}_{{y}^{*}} {\sum }_{y ∈ \left\{0 , 1\right\}} p \left(\left(y\right| \text{x}\right) ⋅ {C}_{y , {y}^{*}}$$

where y∈{0,1} is the class label, and x is the covariate vector (also called feature vector in the machine learning literature). Note that we assume here that c_{0,0} = c_{1,1} = 0. Label 1 denotes the true label, e.g. that the patient has diabetes, whereas label 0 denotes the false label.

Methods described in NPL 1 try to collect only as many covariates as necessary to minimize the total cost of classification, i.e. costs of collecting the covariates + expected costs of misclassification.

For that purpose (NPL 1) assumes a pre-defined sequence of covariates sets S₁ ⊂ S₂ ⊂ S₃ ... Sq. First the covariates in S₁ are acquired, and then depending on the observed values of S₁, either the covariates S₂\S₁ are additionally acquired, or a classification decision is made. In case, the covariates S₂\S₁ are acquired, the same procedure is repeated again analogously. The strategy for either choosing additional covariates or classifying, is such that the total cost of classification is minimized in expectation.

Note that based on each covariate set Si, where i∈2 {1, ... , q}, a classifier is trained returning the probability p(y = 1|x_{Si} ), where x_{Si} is the vector denoting the observed values for covariates Si.

### CITATION LIST

### Non Patent Literature

NPL 1: (Andrade et al, 2019) “Efficient Bayes Risk Estimation for Cost-Sensitive Classification”, Artificial Intelligence and Statistics, 2019.

NPL 2: (Kanao et al, 2009)“PSA CUT-OFF NOMOGRAM THAT AVOID OVER-DETECTION OF PROSTATE CANCER IN ELDERLY MEN”, The Journal of Urology, 2009.

### SUMMARY OF INVENTION

### Technical Problem

A Bayes procedure, and in particular, the method in NPL 1 requires that all misclassification costs are specified. In most situations the misclassification cost c_{0,1} is relatively easy to specify. For example, in the medical domain, it is easier to specify the medical costs for treating a healthy patient who has no diabetes but is wrongly classified as having diabetes.

On the other hand, it is more difficult to specify c_{1,0}. For example, it is more difficult to monetize the exact cost of the case in which a diabetes patient died although he might had been rescued. Therefore, in the medical domain, it is more common to try to make a guarantee on the recall. The terminology “recall” in the machine learning field can be used herein although the term “sensitivity” is more common than “recall” in the medical field. In particular, it is common practice to require that the recall is 95% (see e.g. NPL2).

However, as mentioned above, a Bayes procedure requires the specification of c_{1,0} and cannot make guarantees on the required recall.

The present disclosure has been accomplished to solve the above problems and an object of the present disclosure is thus to provide an information processing apparatus, etc., capable of determining thresholds of a classification procedure which can ensure a user-specified recall.

### Solution to Problem

An information processing apparatus according to the present disclosure is an information processing apparatus for determining a threshold on classification scores, including:
a score ranking component that sorts all classification scores from samples of an evaluation data set that was not used for training the classifier and removes scores for which the class label is false; and
an iteration component that iterates the threshold from the highest score returned from the score ranking component down until the number of samples with score not lower than the current threshold is larger than a user specified recall value times the number of true labels in the evaluation data set.

A method according to the present disclosure is a method for determining a threshold on classification scores, including:
sorting all classification scores from samples of an evaluation data set that was not used for training the classifier and removing scores for which the class label is false; and
iterating the threshold from the highest score returned from the score ranking component down until the number of samples with score not lower than the current threshold is larger than a user specified recall value times the number of true labels in the evaluation data set.

A non-transitory computer readable medium according to the present disclosure is a non-transitory computer readable medium storing a program for causing a computer to execute a method for determining a threshold on classification scores, the method comprising:
sorting all classification scores from samples of an evaluation data set that was not used for training the classifier and removing scores for which the class label is false; and
iterating the threshold from the highest score returned from the score ranking component down until the number of samples with score not lower than the current threshold is larger than a user specified recall value times the number of true labels in the evaluation data set.

### Advantageous Effects of Invention

The present disclosure can determine threshold t to guarantee that, in expectation, the recall of a classification procedure is at least as large as a user-specified value r.

### BRIEF DESCRIPTION OF DRAWINGS

FIG. 1 is the configuration diagram of the threshold estimation apparatus for determining threshold according to a first embodiment of the present disclosure.

FIG. 2 is a diagram illustrating an example of the threshold determination when there is one classifier.

FIG. 3 is a diagram illustrating an example of the threshold determination when there is one classifier.

FIG. 4 is the configuration diagram of the determination apparatus for determining false negative misclassification costs according to a second embodiment of the present disclosure.

FIG. 5 is a diagram illustrating an example of the threshold determination when there is more than one classifier.

FIG. 6 is a diagram illustrating an example of the threshold determination when there is more than one classifier.

FIG. 7 is a diagram illustrating an example of the threshold determination when there is more than one classifier.

FIG. 8 is a diagram illustrating an example of the threshold determination when there is more than one classifier.

FIG. 9 is a diagram illustrating an example of the threshold determination when there is more than one classifier.

FIG. 10 is a diagram illustrating an example of the threshold determination when there is more than one classifier.

FIG. 11 is a block diagram illustrating the configuration example of the estimation apparatus and determination apparatus.

### DESCRIPTION OF EMBODIMENTS

Example exemplary embodiments according to the present disclosure will be described hereinafter with reference to the drawings.

For the clarification of the description, the following description and the drawings may be omitted or simplified as appropriate. Further, each element shown in the drawings as functional blocks that perform various processing can be formed of a CPU (Central Processing Unit), a memory, and other circuits in hardware and may be implemented by programs loaded into the memory in software. Those skilled in the art will therefore understand that these functional blocks may be implemented in various ways by only hardware, only software, or the combination thereof without any limitation. Throughout the drawings, the same components are denoted by the same reference signs and overlapping descriptions will be omitted as appropriate.

Instead of requiring the specification of the misclassification cost c_{1,0}, the present disclosure allows the usage of a user-specified recall r, e.g. r = 95%.

In order to guarantee that the recall of the classification procedure is at least r, the present disclosure calculates a threshold t on the classification probability p(y = 1|x) based on the empirical estimate on hold-out data (=evaluation data). The threshold t output by the present disclosure is only as small as necessary to guarantee a recall of at least r. For example a threshold of 0 would trivially lead to 100% recall, but would have 0% precision.

Furthermore, the acquired threshold t and a user specified false positive cost c_{0,1} allow the calculation of the false negative cost c_{1,0} by using the properties of a Bayes procedure.

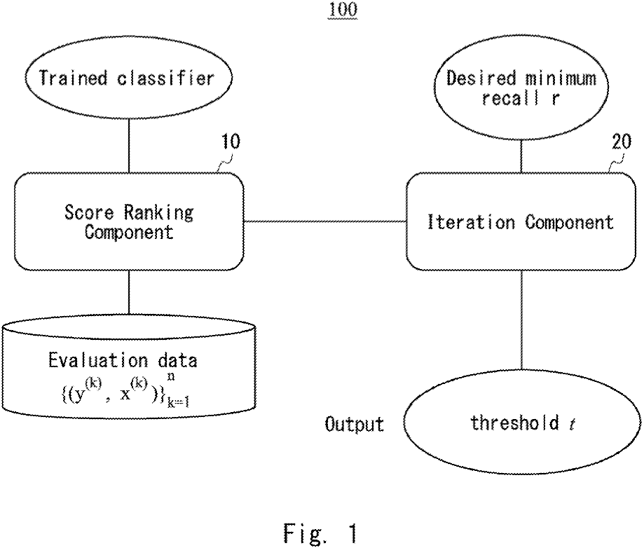

The core components of the threshold estimation apparatus 100 according the first embodiment of the present disclosure are illustrated in FIG. 1 and are explained in the following.

### Mode 1: One Classifier

First, a threshold estimation apparatus according to a first embodiment will be described with reference to FIG. 1. The threshold estimation apparatus 100 according to this embodiment includes score ranking component 10 and iteration component 20. This embodiment shows the simple setting where always all covariates are used for classification.

In the following, the indicator function 1_{M} used herein indicates 1 if expression M is true and otherwise 0. Furthermore, a data set for evaluation with n samples, which is denoted as (y^{(k)}, x^{(k)})ⁿₖ₌₁, can be used.

First, Score Ranking Component 10 can remove all samples for which y^{(k)} = 0, and calculate p^{(k)}₁ := p(y^{(k)} = 1|x^{(k)}), for k = 1,... n_{T} , where n_{T} is the number of true samples (i.e. n_{T} := Σₙ ^{k=1} 1_{y(k)=1}). Furthermore, Score Ranking Component 10 sorts the entries in increasing order and removes all duplicates, thus the resulting sequence is denoted as {p(k)}^{nH}ₖ₌₁.

Next, Iteration Component 20 can perform the following steps outlined in Algorithm 1.

Algorithm 1: Determine threshold t for one classifier.

$$\begin{matrix}m : = {n}_{H} \\ t : = {p}^{\left(m\right)} \\ \text{while} \frac{1}{{n}_{T}} {\sum }_{k}^{{n}_{T}} {}_{\left|\right)} {1}_{{}_{p 1}^{\left(k\right)} \geq t} \leq r \text{do} \\ \left|\begin{matrix}m : = m - 1 \\ t : = {p}^{\left(m\right)}\end{matrix}\right) \\ \text{end} \\ \text{Output} : t\end{matrix}$$

Using the threshold t output by Algorithm 1, the classifier defined by

$$δ \left(\text{x}\right) : = \left\{\begin{matrix}1 & \text{if} p \left(y = 1 \left|\text{x}\right)\right) \geq t . \\ 0 & \text{else} \text{.}\end{matrix}\right)$$

is guaranteed, in expectation, to have recall of at least r.

This can be seen as follows. Given a distribution over (y, x) such that [1_{y=1}] > 0, the recall of a classifier δ is defined as:

$${R}_{δ} : = \int p \left(x \left|y\right) = 1\right) ⋅ {1}_{δ \left(x\right)} {}_{1} d x .$$

Since p(x|y = 1) is unknown, the evaluation data (y(k), x^{(k)})ⁿₖ₌₁ is used to estimate R_{δ}:

$$\begin{matrix}{R}_{δ t} = \int p \left(\left(\text{x}\right| y = 1\right) ⋅ {1}_{δ \left(x\right) = 1} d \text{x} \\ = \int p \left(\text{x} \left|y = 1\right)\right) ⋅ {1}_{p \left(y = 1 \left|\text{x}\right)\right) \geq {}_{t} d x} \\ \approx \frac{1}{{n}_{T}} {\sum }_{k : {y}^{\left(k\right)} 1} {1}_{p \left(y = 1 \left|{\text{x}}^{\left(k\right)}\right)\right) \geq t} \\ = \frac{1}{{n}_{T}} {\sum }_{k : {y}^{\left(k\right)} = 1} {1}_{p \left(y = 1 \left|{\text{x}}^{\left(k\right)}\right)\right) \geq {p}^{\left(m\right)}} \\ = \frac{1}{{n}_{T}} {\sum }_{k = 1}^{{n}_{T}} {1}_{{p}^{\left(k\right)} \geq {p}^{\left(m\right)}} > r ,\end{matrix}$$

where t = p(m), with m being the value after exiting the while loop in Algorithm 1.

As described above, the iteration component 20 (Corresponding to Algorithm 1) that iterates the threshold from the highest score returned from the score ranking component down until the number of samples with score not lower than the current threshold is larger than a user specified recall value times the number of true labels in the evaluation data set.

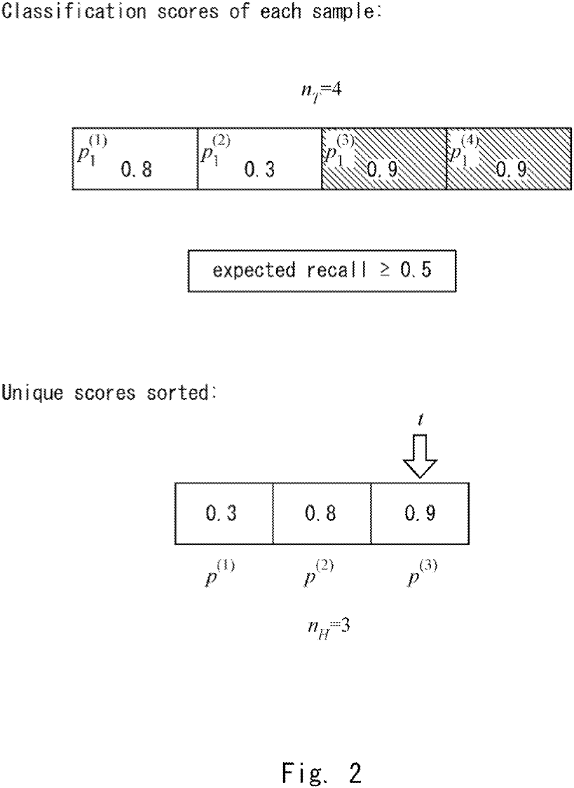

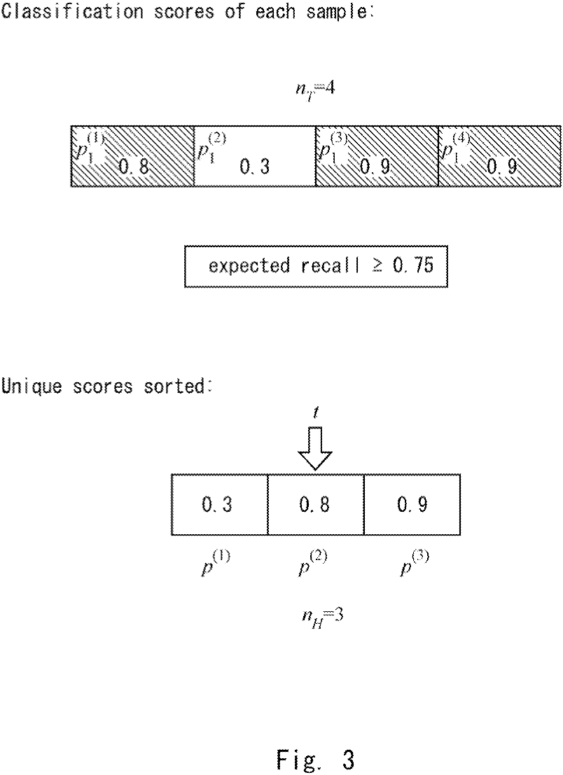

Finally, examples in FIGS. 2 and 3 will be described. FIG. 2 shows the evaluated scores of the samples that have true label (i.e. y = 1), and the unique sorted probabilities of all samples which true class label is 1. In FIG. 2, Classification scores of each sample are 0.8, 0.3, 0.9, 0.9. After removal of duplicates (e.g. 0.9 in FIG. 2), unique sorted scores are 0.3, 0.8, 0.9. First, the threshold for classification is set to 0.9 (the highest classification score). The hatched cells correspond to the number of samples (e.g. 0.9 in FIG. 2) which are correctly classified as true (y = 1) by the classifier. Therefore, the number of correctly classified samples is two (out of four samples which true class label is 1). Accordingly, the expected recall is >= 0.5.

Next, the threshold for classification is lowered to 0.8 (i.e. the second highest score classification score), FIG. 3 shows the evaluated scores of the samples that have true label (i.e. y = 1), and the unique sorted probabilities. The hatched cells correspond to the number of samples (e.g. 0.8 and 0.9 in FIG. 3) which are correctly classified as true (y = 1) by the classifier. Therefore, the number of correctly classified samples is three (out of four samples which true class label is 1). Accordingly, the expected recall is >= 0.75.

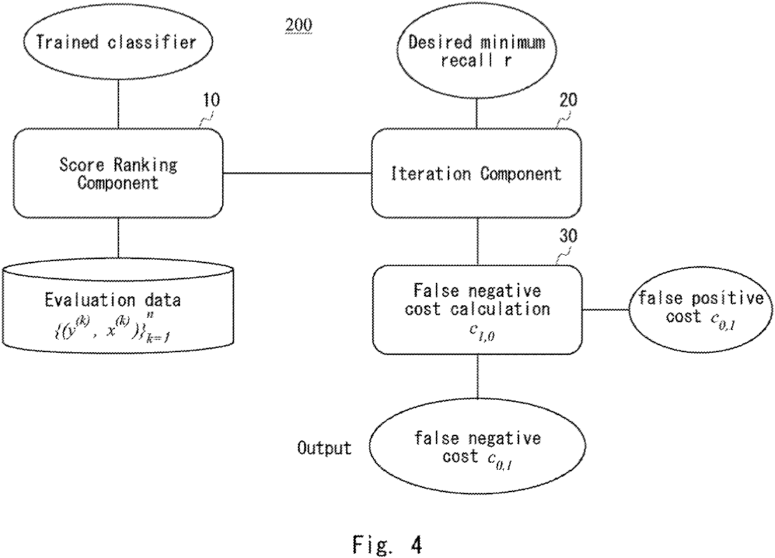

The threshold value t starts at 0.9 in FIG. 3, and goes down till threshold value 0.8 in FIG. 4. The number of hatched cells corresponds to the number of samples which are correctly classified as true (y = 1) by the classifier, when the threshold value is t. If it is assumed that the user specified recall is 0.7, the procedure will exit with threshold 0.8.

### Mode 2: Two or More Classifiers

Next, we consider the situation, where we are given q score functions p(y =1 |x_{Si}), for each i∈{1 ,..., q} and one score function is chosen according to some strategy, e.g. the selection strategy outlined in (NPL1). Here, we do not make any assumptions on the selection strategy.

The threshold for defining classifier δ_{ti,Si}(x_{Si}) is denoted as ti. The classifier δ_{ti,Si}(x_{Si}) is as follows:

$${d}_{{t}_{i} , {S}_{i}} \left({\text{x}}_{{S}_{i}}\right) : = \left\{\begin{matrix}1 & \text{if} p \left(y = 1 \vert {\text{x}}_{{S}_{i}}\right) \geq {t}_{i} , \\ 0 & \text{else} \text{.}\end{matrix}\right)$$

In the following, the thresholds tᵢ can be found such that the following requirement is satisfied:

$$p \left({δ}_{{t}_{1} , {S}_{1}} = 1 , {δ}_{{t}_{2 ,} {S}_{2}} = 1 , … , {δ}_{{t}_{q} , {S}_{q}} = 1 \vert y = 1\right) \geq r .$$

When that the above inequality is satisfied, it is ensured that the recall of any classier selection strategy is at least r. The reason is as follows. Assume that the label of the sample is true (i.e. y = 1) and any classifier δ_{ti,Si} outputs label 0, then an adversarial selection strategy (a selection strategy which tries to produce the lowest recall) will select this classifier. Otherwise, if all classifiers output label 1, then even an adversarial selection strategy needs to select a classifier δ_{ti,Si} for which the output is 1. By the requirement of Inequality (2), the selecting a classifier δ_{ti,Si} for which the output is 1 will happen with probability of at least r.

As described before, a data set for evaluation with n samples is denoted as (y^{(k)}, x^{(k)})ⁿ_{k=1.} Without loss of generality, we assume that the samples are sorted such that all positive samples (i.e. y = 1) come first, that is yₖ = 1 for k∈1, 2,..., n_{T} , where n_{T} denotes the total number of positive samples.

For each classifier i∈ {1,..., q}, Threshold estimation apparatus 100 determines a threshold tᵢ as follows:

First, Score ranking component 10 calculates p^{(k)}ᵢ := p(y^{(k)} = 1|x^{(k)}_{Si} ), for k = 1,..., n_{T}. Next, for each classifier i, Score ranking component 10 sorts p^{(k)}ᵢ in increasing order and removes duplicates. The resulting sequence is denoted as {1^{(m)}ᵢ}^{nHi}m=1.

Iteration Component 20 then performs the following steps described in Algorithm 2.

Algorithm 2: Determine thresholds ti for different classifiers.

$$\begin{matrix}\text{for} i ∈ 1 , 2 , … , q \text{do} \\ {m}_{i} : = {n}_{{H}_{1}} \\ {t}_{i} : = {l}_{i}^{\left({m}_{i}\right)} \\ \text{end} \\ \text{while} i n e q u a l i t y \left(2\right) n o t f u l l f i l l e d \text{do} \\ i * : = arg {max}_{i ∈ 1 , 2 , … , q} {m}_{i} \\ {m}_{i *} : = {m}_{i *} - 1 \\ {t}_{i *} : = {l}_{i *}^{\left({m}_{i *}\right)} \\ \text{end} \\ \text{Output:} {\left\{{t}_{i}\right\}}_{i = 1}^{q}\end{matrix}$$

For evaluating inequality (2), Iteration Component 20 uses the empirical estimate of p(δ_{t1,S1} =1, δ_{t2,S2} = 1,..., δ_{tq,Sq} = 1|y = 1), that is

$$\begin{matrix}p \left({δ}_{{t}_{1 ,} {S}_{1}} = 1 , {δ}_{{t}_{2 ,} {S}_{2}} = 1 , … , {δ}_{{t}_{q} , {S}_{q}} = 1 \vert y = 1\right) \approx \\ \frac{1}{{n}_{T}} {\sum }_{k = 1}^{{n}_{T}} {∀ i ∈ \left\{1 , … , q\right\} : {p}_{i}^{\left({k}_{i}\right)} \geq {t}_{i}}_{} .\end{matrix}$$

Note that the while loop in the above algorithm necessarily exits, since eventually for all i∈1, 2, ... , q, we have tᵢ = p⁽¹⁾ᵢ, where p⁽¹⁾ᵢ is the smallest score in the evaluation data for classifier i.

Furthermore, Threshold estimation apparatus 100 determines the thresholds are only as large as necessary to guarantee that, in expectation, the recall is at least r.

### Simplification for Common Threshold

It is noted that the above procedure performed by Threshold estimation apparatus 100 can be simplified (and speed-up), if it is required that all thresholds tᵢ are the same, which is denoted as t.

First, score ranking component 10 as shown in FIG. 1 places all probabilities p^{(k)}ᵢ, for k∈{1,..., n_{T}}, and i∈{1,..., q} in one array. Next, score ranking component 10 removes duplicates and sorts the entries in increasing order, where the resulting sequence is denoted as {p(k)}^{nH}ₖ₌₁.

Furthermore, let

$${B}_{k} : = \left\{\begin{matrix}1 & \text{if} {\sum }_{i = 1}^{q} {{p}_{i}^{\left(k\right)} \geq l}_{} = q , \\ 0 & \text{else} \text{.}\end{matrix}\right)$$

which indicates whether sample k is correctly classified as y = 1 by all classifiers, when we assume threshold t.

The iteration component 20 as shown in FIG. 1 then determines threshold t using Algorithm 3.

Algorithm 3: Determine threshold t common to different classifiers.

$$\begin{matrix}m : = {n}_{H} \\ t : = {p}^{\left(m\right)} \\ \text{while} \frac{{\sum }_{k m}^{{n}_{T}} B}{{n}_{T}} \leq r \text{do} \\ \left|\begin{matrix}m : = m - 1 \\ t : = p \left(m\right)\end{matrix}\right) \\ \text{end} \\ \text{Output:} t\end{matrix}$$

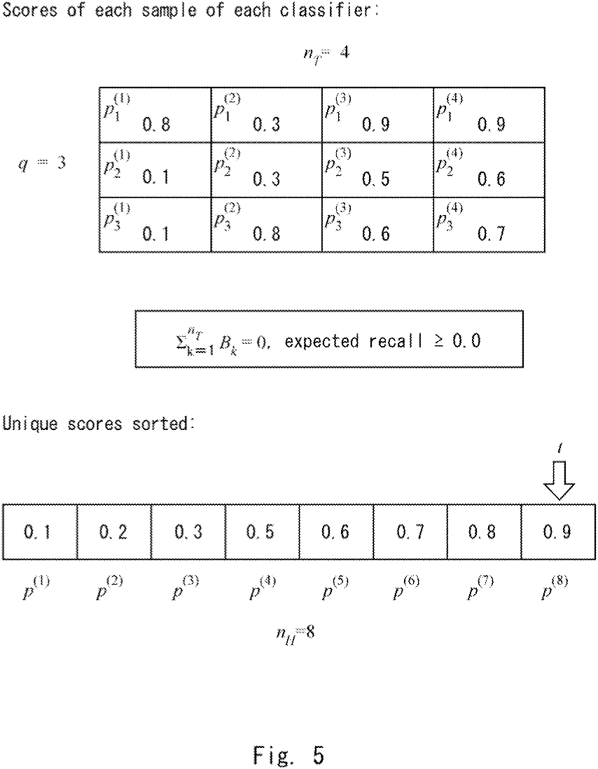

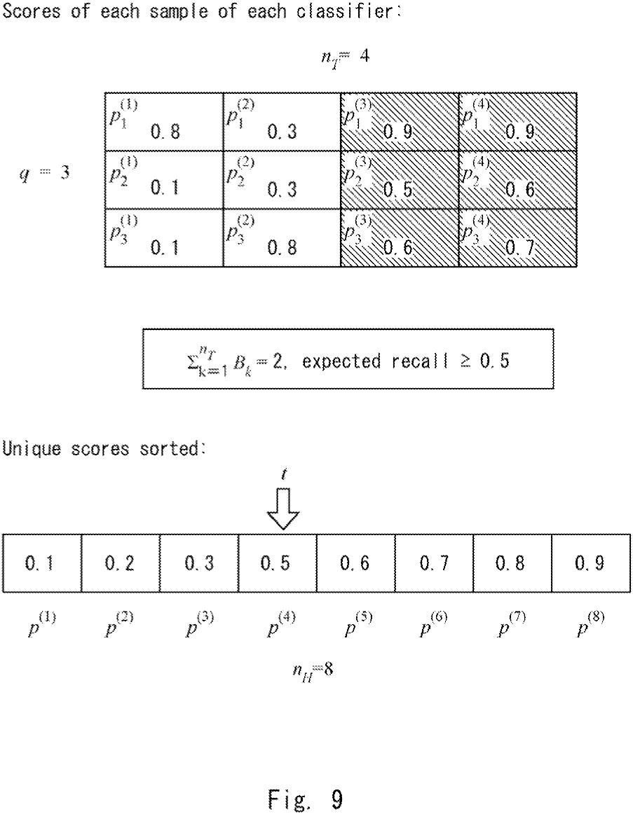

Finally, examples in FIGS. 5 to 10 will be described. FIG. 5 shows the evaluated scores of the samples that have true label (i.e. y = 1), and the unique sorted probabilities. Note that in the matrix, each row corresponds to the scores of one classifier, and each column corresponds to one sample. The first threshold value starts at 0.9, and goes down till threshold value 0.3. The number of hatched columns corresponds to the number of samples which are correctly classified as true by all classifiers, when the threshold value is t. If we assume that the user specified recall is 0.7, the procedure will exit with threshold 0.3. In more detail, first, in FIG. 5, the threshold is set to t = 0.9, the highest score (of all scores returned by all classifiers). In this case no sample is classified as true by all classifiers. Therefore,

$${\sum }_{\text{k=1}}^{{\text{n}}_{\text{T}}} {\text{B}}_{\text{k}}$$

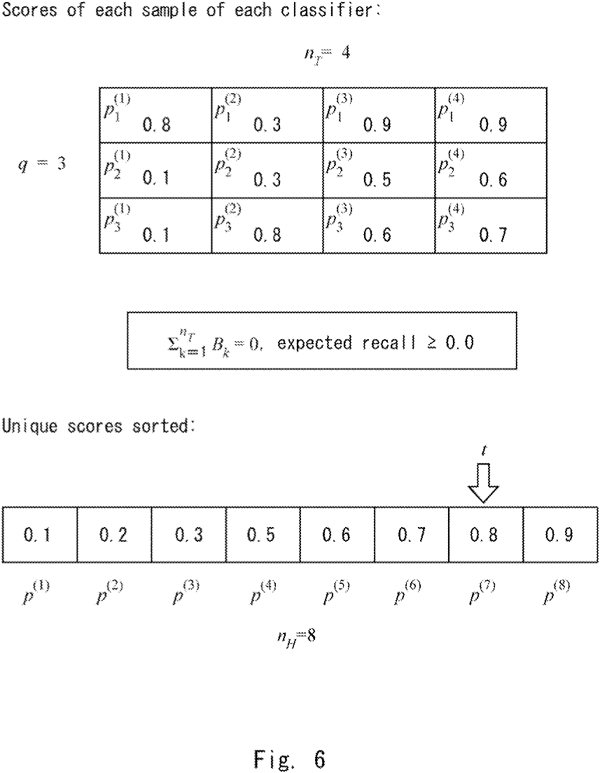

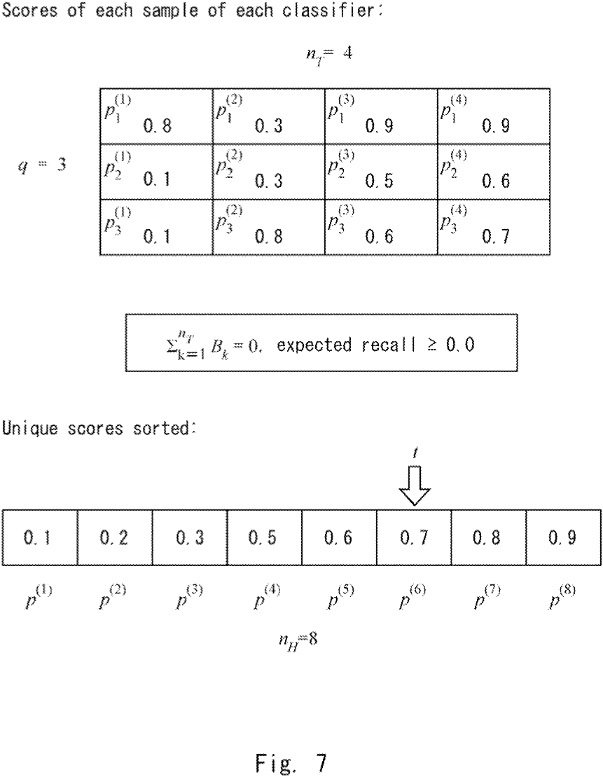

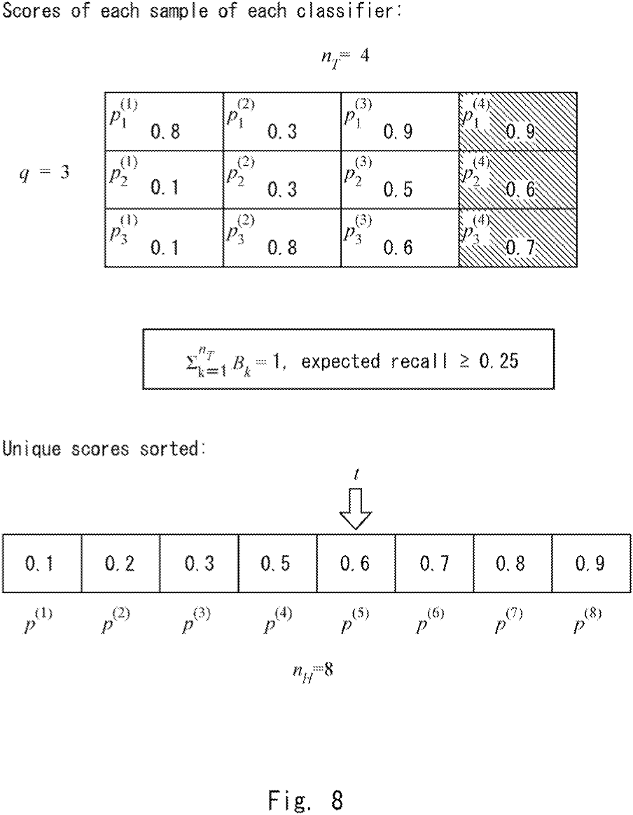

(the number of samples which are classified correctly by all classifiers) is 0. Next, in FIG. 6, the threshold is lowered to 0.8. Since the classification result does not change, also stays 0. This procedure is continued in FIGS. 7, 8,9,10. Where in FIG. 8, the threshold t is lowered to 0.6, and thus, for the first time all classifiers classify sample (4) correctly, and therefore

$${\sum }_{\text{k=1}}^{{\text{n}}_{\text{T}}} {\text{B}}_{\text{k}}$$

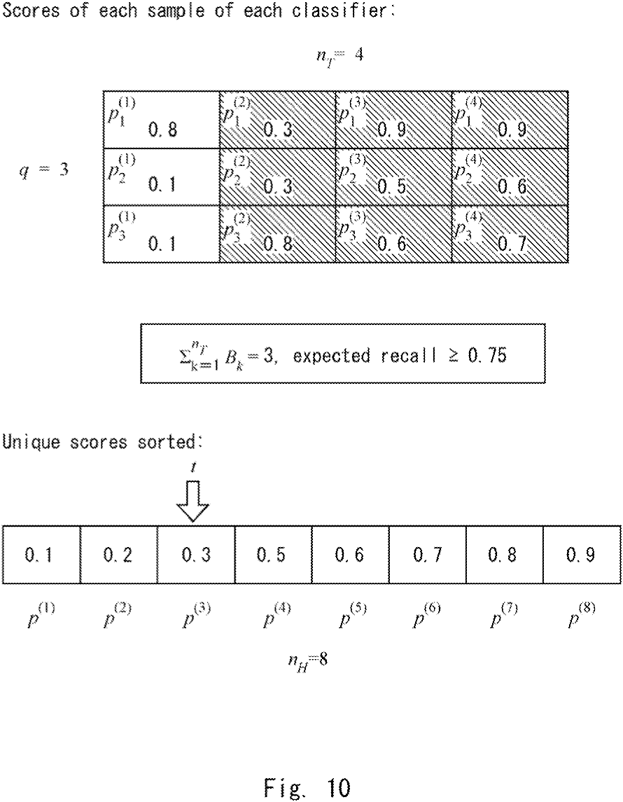

becomes 1. Finally, in FIG. 10, the threshold t is lowered to 0.3, and

$${\sum }_{\text{k=1}}^{{\text{n}}_{\text{T}}} {\text{B}}_{\text{k}} = 3$$

, and since

$$\frac{{\sum }_{\text{k=1}}^{{\text{n}}_{\text{T}}} {\text{B}}_{\text{k}}}{{n}_{T}} = 0.75 > = 0.7$$

(the user specified threshold), the procedure ends, and returns threshold t = 0.3.

As described above, the iteration component 20 stops the iteration until the number of times where all scores corresponding to one sample but from different classifiers are larger than the threshold, is larger than a user specified recall value times the number of true labels in the evaluation data (Corresponding to

$${\sum }_{\text{k=1}}^{{\text{n}}_{\text{T}}} {\text{B}}_{\text{k}} > {n}_{T} ⋅ r$$

).

### Mode 3: Application to Cost Sensitive Classification

Finally, the threshold t as determined using Algorithm 1 and Algorithm 3 can be used to determine the false negative cost c_{1,0}. The false negative cost c_{1,0} is used to define a Bayes Classifier.

The complete Diagram of false negative cost determination apparatus 200 is shown in FIG. 4. The false negative cost determination apparatus 200 includes Score ranking component 10, Iteration component 20, and False negative cost calculation component 30.

Assuming that classifier δ is a Bayes classifier, False negative cost calculation component 30 (see FIG. 4) which is set as

$${C}_{1 , 0} : = \frac{1 - t}{t} {C}_{0 , 1}$$

ensures that the recall of classifier is at least r. Specifically, the false negative misclassification cost is determined by the reciprocal of the threshold minus 1, and the resulting value multiplied by the false positive misclassification cost which is assumed to be provided by the user. The reason is as follows. Assuming that is a Bayes procedure (see definition in Equation 1), we have

$$\begin{matrix}δ \left(\text{x}\right) = 1 ⇔ p \left(y = 1 \vert \text{x}\right) ⋅ {c}_{1 , 0} \geq p \left(y = 0 \vert \text{x}\right) ⋅ {c}_{0 , 1} \\ ⇔ p \left(y = 1 \vert \text{x}\right) ⋅ {c}_{1 , 0} \geq \left(1 - p \left(y = 1 \vert \text{x}\right)\right) ⋅ {c}_{0 , 1} \\ ⇔ p \left(y = 1 \vert \text{x}\right) \geq \frac{{c}_{1 , 0}}{{c}_{1 , 0 + {c}_{0 , 1}}} \\ ⇔ p \left(y = 1 \vert \text{x}\right) \geq t .\end{matrix}$$

Therefore, the false negative cost determination apparatus 200 can obtain the recall of a classifier δ as follows.

$${R}_{δ} = \int p \left(\text{x} \left|y = 1\right)\right) ⋅ {1}_{p \left(y = 1 \left|\text{x}\right)\right) \geq t} d \text{x} \text{.}$$

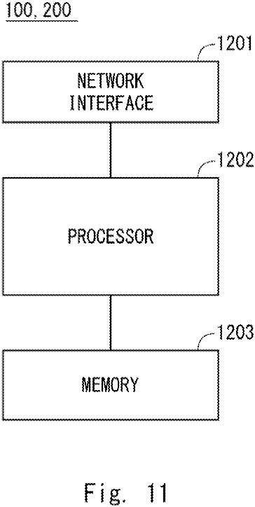

FIG. 11 is a block diagram illustrating the configuration example of the estimation apparatus and determination apparatus. In view of FIG. 11, the estimation apparatus 100 and determination apparatus 200 includes a network interface 1201, a processor 1202 and a memory 1203. The network interface 1201 is used to communicate with a network node (a remote node 10 and the core network 40). The network interface 1201 may include, for example, a network interface card (NIC) compliant with, for example, IEEE 802.3 series.

The processor 1202 performs processing of a center node 20 described with reference to the sequence diagrams and the flowchart in the above embodiments by reading software (computer program) from the memory 1203 and executing the software. The processor 1202 may be, for example, a microprocessor, an MPU or a CPU. The processor 1202 may include a plurality of processors.

The processor 1202 performs data plane processing which includes digital baseband signal processing for wireless communication, and control plane processing. In a case of, for example, LTE and LTE-Advanced, the digital baseband signal processing of the processor 1004 may include signal processing of a PDCP layer, an RLC layer and an MAC layer. Furthermore, the signal processing of the processor 1202 may include signal processing of a GTP-U•UDP/IP layer in an X2-U interface and an S1-U interface. Furthermore, the control plane processing of the processor 1004 may include processing of an X2AP protocol, an S1-MME protocol and an RRC protocol.

The processor 1202 may include a plurality of processors. For example, the processor 1004 may include a modem processor (e.g., DSP) which performs the digital baseband signal processing, a processor (e.g. DSP) which performs the signal processing of the GTP-U•UDP/IP layer in the X2-U interface and the S1-U interface, and a protocol stack processor (e.g., a CPU or an MPU) which performs the control plane processing.

The memory 1203 is configured by a combination of a volatile memory and a non-volatile memory. The memory 1203 may include a storage disposed apart from the processor 1202. In this case, the processor 1202 may access the memory 1203 via an unillustrated I/O interface.

In the example in FIG. 11, the memory 1203 is used to store a software module group. The processor 1202 can perform processing of the estimation apparatus and the determination apparatus described in the above embodiments by reading these software module groups from the memory 1203 and executing the software module groups.

In the above-described exemplary embodiment, the programs may be stored in various types of non-transitory computer readable media and thereby supplied to computers. The non-transitory computer readable media includes various types of tangible storage media.

Examples of the non-transitory computer readable media include a magnetic recording medium (such as a flexible disk, a magnetic tape, and a hard disk drive) and a magneto-optic recording medium (such as a magneto-optic disk).

Further, examples of the non-transitory computer readable media include CD-ROM (Read Only Memory), CD-R, and CD-R/W. Further, examples of the non-transitory computer readable media include a semiconductor memory. The semiconductor memory includes, for example, a mask ROM, a PROM (Programmable ROM), an EPROM (Erasable PROM), a flash ROM, and a RAM (Random Access Memory).

These programs may be supplied to computers by using various types of transitory computer readable media. Examples of the transitory computer readable media include an electrical signal, an optical signal, and an electromagnetic wave. The transitory computer readable media can be used to supply programs to a computer through a wired communication line (e.g., electric wires and optical fibers) or a wireless communication line.

Note that the present disclosure is not limited to the above-described example exemplary embodiments and can be modified as appropriate without departing from the spirit and scope of the present disclosure. Further, the present disclosure may be implemented by combining these example exemplary embodiments as desired.

Although the present disclosure is explained above with reference to example exemplary embodiments, the present disclosure is not limited to the above-described example exemplary embodiments.

### INDUSTRIAL APPLICABILITY

Guaranteeing the recall of a decision procedure (classifier) is important for many risk critical applications. For example in the medical domain it is common to require a minimum value on the recall.

### REFERENCE SIGNS LIST

10 Score Ranking Component
20 Iteration Component
30 False Negative cost Calculation Component
100 Threshold Estimation Apparatus
200 False Negative Cost Determination Apparatus

What is claimed is:

1. An information processing apparatus for determining a threshold on classification scores, comprising:
a score ranking component that sorts all classification scores from samples of an evaluation data set that was not used for training the classifier and removes scores for which the class label is false; and
an iteration component that iterates the threshold from the highest score returned from the score ranking component down until the number of samples with score not lower than the current threshold is larger than a user specified recall value times the number of true labels in the evaluation data set.

2. The information processing apparatus according to claim 1, wherein the score ranking component pools all classification scores from two or more classifiers together before sorting,
and wherein the iteration component stops the iteration until the number of times where all scores corresponding to one sample but from different classifiers are larger than the threshold, is larger than a user specified recall value times the number of true labels in the evaluation data.

3. The information processing apparatus according to claim 1, further comprising a false negative cost calculation component that calculates a false negative misclassification cost, and
wherein the false negative misclassification cost is determined by the reciprocal of the threshold minus 1, and the resulting value multiplied by the false positive misclassification cost.

4. The information processing apparatus according to claim 1, wherein the score ranking component removes duplicate scores.

5. A method for determining a threshold on classification scores, comprising:
sorting all classification scores from samples of an evaluation data set that was not used for training the classifier and removing scores for which the class label is false; and
iterating the threshold from the highest score returned from the score ranking component down until the number of samples with score not lower than the current threshold is larger than a user specified recall value times the number of true labels in the evaluation data set.

6. A non-transitory computer readable medium storing a program for causing a computer to execute a method for determining a threshold on classification scores, the method comprising:
sorting all classification scores from samples of an evaluation data set that was not used for training the classifier and removing scores for which the class label is false; and
iterating the threshold from the highest score returned from the score ranking component down until the number of samples with score not lower than the current threshold is larger than a user specified recall value times the number of true labels in the evaluation data set.
